(19) United States
(12) Patent Application Publication
(10) Pub. No.: US 2005/0277864 A1
(43) Pub. Date: Dec. 15, 2005

(54) Injectable gel implant for glaucoma treatment

(76) Inventors: David Haffner, Mission Viejo, CA (US); Morteza Gharib, San Marino, CA (US); Hosheng Tu, Newport Coast, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/126,868

(22) Filed: May 11, 2005

Related U.S. Application Data
(63) Continuation-in-part of application No. 10/395,633, filed on Mar. 21, 2003, now abandoned.
Application No. 10/395,633 is a continuation of application No. 09/549,350, filed on Apr. 14, 2000, now Pat. No. 6,638,239.

(51) Int. Cl. A61M 5/00

(52) U.S. Cl. 604/8

(57) ABSTRACT

Methods and implants for treating glaucoma in an eye are described. The implant includes an inlet section configured to be positioned in the anterior chamber of the eye and an outlet section in fluid communication with the inlet section. The outlet section is configured to be positioned in Schlemm's canal of the eye. The implant comprises a hydrogel and is configured to conduct aqueous humor from the anterior chamber to Schlemm's canal.

### CLAIM OF PRIORITY AND RELATED APPLICATIONS

This patent application is a continuation-in-part application of U.S. patent application Ser. No. 10/395,633, filed Mar. 21, 2003, which is a continuation application of U.S. patent application Ser. No. 09/549,350, filed Apr. 14, 2000, now U.S. Pat. No. 6,638,239, both of which are incorporated in their entirety by reference herein.

### FIELD OF THE INVENTION

This invention relates generally to methods and devices for reducing intraocular pressure within the animal eye and, more particularly, to methods and devices for expanding Schlemm's canal and/or trabecular meshwork with an injectable implant that solidifies within a target body channel.

### BACKGROUND OF THE INVENTION

The human eye is a specialized sensory organ capable of light reception and is able to receive visual images. Aqueous humor is a transparent liquid that fills the region between the cornea, at the front of the eye, and the lens. A trabecular meshwork, located in an anterior chamber angle formed between the iris and the cornea, serves as a drainage channel for aqueous humor from the anterior chamber, which maintains a balanced pressure within the anterior chamber of the eye.

About two percent of people in the United States have glaucoma. Glaucoma is a group of eye diseases encompassing a broad spectrum of clinical presentations, etiologies, and treatment modalities. Glaucoma causes pathological changes in the optic nerve, visible on the optic disk, and it causes corresponding visual field loss, resulting in blindness if untreated. Lowering intraocular pressure is the major treatment goal in all glaucomas.

In glaucomas associated with an elevation in eye pressure (intraocular hypertension), the source of resistance to outflow is mainly in the trabecular meshwork. The tissue of the trabecular meshwork allows the aqueous humor (hereinafter referred to as “aqueous”) to enter Schlemm's canal, which then empties into aqueous collector channels in the posterior wall of Schlemm's canal and then into aqueous veins, which form the episcleral venous system. Aqueous is continuously secreted by a ciliary body around the lens, so there is a constant flow of aqueous from the ciliary body to the anterior chamber of the eye. Pressure within the eye is determined by a balance between the production of aqueous and its exit through the trabecular meshwork (major route) and uveal scleral outflow (minor route). The portion of the trabecular meshwork adjacent to Schlemm's canal (the juxtacanilicular meshwork) causes most of the resistance to aqueous outflow.

Glaucoma is broadly classified into two categories: closed-angle glaucoma, also known as angle closure glaucoma, and open-angle glaucoma. Closed-angle glaucoma is caused by closure of the anterior chamber angle by contact between the iris and the inner surface of the trabecular meshwork. Closure of this anatomical angle prevents normal drainage of aqueous from the anterior chamber of the eye. Open-angle glaucoma is any glaucoma in which the exit of aqueous through the trabecular meshwork is diminished while the angle of the anterior chamber remains open. For most cases of open-angle glaucoma, the exact cause of diminished filtration is unknown. Primary open-angle glaucoma is the most common of the glaucomas, and is often asymptomatic in the early to moderately advanced stages of glaucoma. Patients may suffer substantial, irreversible vision loss prior to diagnosis and treatment. However, there are secondary open-angle glaucomas which may include edema or swelling of the trabecular spaces (e.g., from corticosteroid use), abnormal pigment dispersion, or diseases such as hyperthyroidism that produce vascular congestion.

All current therapies for glaucoma are directed toward decreasing intraocular pressure. Currently recognized categories of drug therapy for glaucoma include: (1) Miotics (e.g., pilocarpine, carbachol, and acetylcholinesterase inhibitors), (2) Sympathomimetics (e.g., epinephrine and dipivalylepinephxine), (3) Beta-blockers (e.g., betaxolol, levobunolol and timolol), (4) Carbonic anhydrase inhibitors (e.g., acetazolamide, methazolamide and ethoxzolamide), and (5) Prostaglandins (e.g., metabolite derivatives of arachindonic acid). Medical therapy includes topical ophthalmic drops or oral medications that reduce the production of aqueous or increase the outflow of aqueous. However, drug therapies for glaucoma are sometimes associated with significant side effects. The most frequent and perhaps most serious drawback to drug therapy is that patients, especially the elderly, often fail to correctly self-medicate. Such patients forget to take their medication at the appropriate times or else administer eye drops improperly, resulting in under- or over-dosing. Because the effects of glaucoma are irreversible, when patients dose improperly, allowing ocular concentrations to drop below appropriate therapeutic levels, further permanent damage to vision occurs. Furthermore, current drug therapies are targeted to be deposited directly into the ciliary body where the aqueous is produced. In addition, current therapies do not provide for a continuous slow-release of the drug. When drug therapy fails, surgical therapy is pursued.

Surgical therapy for open-angle glaucoma consists of laser trabeculoplasty, trabeculectomy, and implantation of aqueous stents after failure of trabeculectomy or if trabeculectomy is unlikely to succeed. Trabeculectomy is a major surgery that is widely used and is augmented with topically applied anticancer drugs, such as 5-flurouracil or mitomycin-C to decrease scarring and increase the likelihood of surgical success.

Approximately 100,000 trabeculectomies are performed on Medicare-age patients per year in the United States. This number would likely increase if ocular morbidity associated with trabeculectomy may be decreased. The current morbidity associated with trabeculectomy consists of failure (10-15%); infection (a life long risk of 2-5%); choroidal hemorrhage, a severe internal hemorrhage from low intraocular pressure, resulting in visual loss (1%); cataract formation; and hypotony maculopathy (potentially reversible visual loss from low intraocular pressure). For these reasons, surgeons have tried for decades to develop a workable surgery for the trabecular meshwork.

The surgical techniques that have been tried and practiced are goniotomy/trabeculotomy and other mechanical disruptions of the trabecular meshwork, such as trabeculopuncture, goniophotoablation, laser trabecular ablation, and goniocurretage. These are all major operations and are briefly described below.

Goniotomy and trabeculotomy are simple and directed techniques of microsurgical dissection with mechanical disruption of the trabecular meshwork. These initially had early favorable responses in the treatment of open-angle glaucoma. However, long-term review of surgical results showed only limited success in adults. In retrospect, these procedures probably failed due to cellular repair and fibrosis mechanisms and a process of “filling in.” Filling in is a detrimental effect of collapsing and closing in of the created opening in the trabecular meshwork. Once the created openings close, the pressure builds back up and the surgery fails.

Q-switched Neodynium (Nd) YAG lasers also have been investigated as an optically invasive trabeculopuncture technique for creating full-thickness holes in trabecular meshwork. However, the relatively small hole created by this trabeculopuncture technique exhibits a filling-in effect and fails.

Goniophotoablation is disclosed by Berlin in U.S. Pat. No. 4,846,172 and involves the use of an excimer laser to treat glaucoma by ablating the trabecular meshwork. This method did not succeed in a clinical trial. Hill et al. used an Erbium YAG laser to create full-thickness holes through trabecular meshwork (Hill et al., Lasers in Surgery and Medicine 11:341-346, 1991). This laser trabecular ablation technique was investigated in a primate model and a limited human clinical trial at the University of California, Irvine. Although ocular morbidity was zero in both trials, success rates did not warrant further human trials. Failure was again from filling in of surgically created defects in the trabecular meshwork by repair mechanisms. Neither of these is a viable surgical technique for the treatment of glaucoma.

Goniocurretage is an “ab interno” (from the inside), mechanically disruptive technique that uses an instrument similar to a cyclodialysis spatula with a microcurrette at the tip. Initial results were similar to trabeculotomy: it failed due to repair mechanisms and a process of filling in.

Although trabeculectomy is the most commonly performed filtering surgery, viscocanulostomy (VC) and non penetrating trabeculectomy (NPT) are two new variations of filtering surgery. These are “ab externo” (from the outside), major ocular procedures in which Schlemm's canal is surgically exposed by making a large and very deep scleral flap. In the VC procedure, Schlemm's canal is cannulated and viscoelastic substance injected (which dilates Schlemm's canal and the aqueous collector channels). In the NPT procedure, the inner wall of Schlemm's canal is stripped off after surgically exposing the canal.

Trabeculectomy, VC, and NPT involve the formation of an opening or hole under the conjunctiva and scleral flap into the anterior chamber, such that aqueous is drained onto the surface of the eye or into the tissues located within the lateral wall of the eye. These surgical operations are major procedures with significant ocular morbidity. When trabeculectomy, VC, and NPT are thought to have a low chance for success, a number of implantable drainage devices have been used to ensure that the desired filtration and outflow of aqueous through the surgical opening will continue. The risk of placing a glaucoma drainage device also includes hemorrhage, infection, and diplopia (double vision).

Examples of implantable stents and surgical methods for maintaining an opening for the release of aqueous from the anterior chamber of the eye to the sclera or space beneath the conjunctiva have been disclosed in, for example, Hsia et al., U.S. Pat. No. 6,059,772 and Baerveldt, U.S. Pat. No. 6,050,970.

All of the above embodiments and variations thereof have numerous disadvantages and moderate success rates. They involve substantial trauma to the eye and require great surgical skill in creating a hole through the full thickness of the sclera into the subconjunctival space. The procedures are generally performed in an operating room and involve a prolonged recovery time for vision. The complications of existing filtration surgery have prompted ophthalmic surgeons to find other approaches to lowering intraocular pressure.

Because the trabecular meshwork and juxtacanilicular tissue together provide the majority of resistance to the outflow of aqueous, they are logical targets for surgical removal in the treatment of open-angle glaucoma. In addition, minimal amounts of tissue need be altered and existing physiologic outflow pathways may be utilized.

As reported in Arch. Ophthalm. (2000) 118:412, glaucoma remains a leading cause of blindness, and filtration surgery remains an effective, important option in controlling glaucoma. However, modifying existing filtering surgery techniques in any profound way to increase their effectiveness appears to have reached a dead end. The article further states that the time has come to search for new surgical approaches that may provide better and safer care for patients with glaucoma.

### SUMMARY OF THE INVENTION

Accordingly, there is a need for site-specific treatment methods for diverting aqueous humor from the anterior chamber into Schlemm's canal. Disclosed herein are methods and devices for reducing intraocular pressure within the eye and, more particularly, for expanding Schlemm's canal and/or trabecular meshwork with an injectable foam implant that solidifies within a target body channel. In some embodiments disclosed herein, an injectable foam implant is provided with at least a portion sized and configured to expand after implantation. The foam implant is preferably adapted for retention within Schlemm's canal or other body opening.

In some preferred embodiments, an implantable seton has an inlet portion configured to extend through a portion of the trabecular meshwork of an eye, and an outlet portion configured to extend into Schlemm's canal of the eye, wherein the inlet portion is disposed at an angle relative to the outlet portion. In some embodiments, the outlet portion has a lumen with an oval cross-section having a long axis.

The outlet portion in certain embodiments has a longitudinal axis, such that the long axis of the oval cross-section and the longitudinal axis of the outlet portion define a plane, the inlet portion having a longitudinal axis which lies outside the plane at an angle θ (theta) thereto.

In some preferred arrangements, the seton comprises an inlet portion, configured to extend through a portion of the trabecular meshwork; an outlet portion, configured to extend into Schlemm's canal; and at least one protrusion on the outlet portion, configured to exert traction against an inner surface of Schlemm's canal. This protrusion can comprise at least one barb or ridge.

Some preferred embodiments comprise an inlet portion configured to extend through a portion of the trabecular meshwork, an outlet portion configured to extend into Schlemm's canal, and a one-way valve within the inlet and/or outlet portions.

A method for delivering a seton within an eye is disclosed, comprising providing an elongate guide member, advancing a distal end of the guide member through at least a portion of the trabecular meshwork of the eye, advancing the seton along the guide member toward the distal end, and positioning the seton to conduct aqueous humor between the anterior chamber of the eye and Schlemm's canal.

In certain embodiments, the advancing of the guide member comprises advancing it from the anterior chamber into the trabecular meshwork. In further embodiments, the positioning comprises positioning an end of the seton within Schlemm's canal adjacent to an aqueous collection channel.

Certain preferred embodiments include an apparatus for delivering a seton to the anterior chamber of an eye comprising an elongate tube having a lumen, an outer surface, and a distal end; a removable, elongate guide member within the lumen, configured to permit the seton to be advanced and to be positioned in the trabecular meshwork of the eye. This apparatus can further comprise a cutting member positioned at the distal end of the tube. The cutting member can be selected from the group consisting of a knife, a laser probe, a pointed guide member, a sharpened distal end of said tube, and an ultrasonic cutter. The apparatus can also further comprise an opening in the outer surface of the tube, configured to allow fluid infusion into the eye.

In further preferred embodiments, an apparatus for delivering a seton in an eye, comprises an elongate member adapted for insertion into an anterior chamber of the eye, the elongate member having a distal end portion configured to retain the seton therein, the distal end portion comprising a cutting member configured to form an opening in the trabecular meshwork of the eye for receipt of the seton, such that one end of the seton is in Schlemm's canal. The elongate member can further comprise a lumen which conducts fluid toward said distal end portion.

The preferred embodiment provides further surgical treatment of glaucoma (trabecular bypass surgery) at the level of trabecular meshwork and restores existing physiological outflow pathways. An implant bypasses diseased trabecular meshwork at the level of trabecular meshwork and which restores existing physiological outflow pathways. The implant has an inlet end, an outlet end and a lumen therebetween. The inlet is positioned in the anterior chamber at the level of the internal trabecular meshwork and the outlet end is positioned at about the exterior surface of the diseased trabecular meshwork and/or into fluid collection channels of the existing outflow pathways.

In accordance with a preferred method, trabecular bypass surgery creates an opening or a hole through the diseased trabecular meshwork through minor microsurgery. To prevent “filling in” of the hole, a biocompatible elongated implant is placed within the hole as a seton, which may include, for example, a solid rod or hollow tube. In one exemplary embodiment, the seton implant may be positioned across the diseased trabecular meshwork alone and it does not extend into the eye wall or sclera. In another embodiment, the inlet end of the implant is exposed to the anterior chamber of the eye while the outlet end is positioned at the exterior surface of the trabecular meshwork. In another exemplary embodiment, the outlet end is positioned at and over the exterior surface of the trabecular meshwork and into the fluid collection channels of the existing outflow pathways. In still another embodiment, the outlet end is positioned in the Schlemm's canal. In an alternative embodiment, the outlet end enters into fluid collection channels up to the level of the aqueous veins with the seton inserted in a retrograde or antegrade fashion.

According to the preferred embodiment, the seton implant is made of biocompatible material, which is either hollow to allow the flow of aqueous humor or solid biocompatible material that imbibes aqueous. The material for the seton may be selected from the group consisting of porous material, semi-rigid material, soft material, hydrophilic material, hydrophobic material, hydrogel, elastic material, and the like.

In further accordance with the preferred embodiment, the seton implant may be rigid or it may be made of relatively soft material and is somewhat curved at its distal section to fit into the existing physiological outflow pathways, such as Schlemm's canal. The distal section inside the outflow pathways may have an oval shape to stabilize the seton in place without undue suturing. Stabilization or retention of the seton may be further strengthened by a taper end and/or by at least one ridge or rib on the exterior surface of the distal section of the seton, or other surface alterations designed to retain the seton.

In one embodiment, the seton may include a micropump, one way valve, or semi-permeable membrane if reflux of red blood cells or serum protein becomes a clinical problem. It may also be useful to use a biocompatible material that hydrates and expands after implantation so that the seton is locked into position around the trabecular meshwork opening or around the distal section of the seton.

In one embodiment, a porous foam implant is adapted for implantation within Schlemm's canal or the trabecular meshwork of the eye such that aqueous humor controllably flows from the anterior chamber of the eye to Schlemm's canal, bypassing the trabecular meshwork. In one embodiment, the porous foam implant comprises a therapeutic agent effective in treating glaucoma, which agent is controllably released from the device into tissue of the trabecular meshwork and/or Schlemm's canal. Depending upon the specific treatment contemplated, therapeutic agents (such as pharmaceuticals, genes, cells, proteins, and/or growth factors) may be utilized in conjunction with the porous foam implant. Placement of the porous foam implant within the eye and incorporation, and eventual release, of a proven therapeutic agent may inhibit or slow the effects of glaucoma.

In another embodiment, a porous foam implant is provided that is implantable within an eye. The implant comprises injectable liquid material that is controllably solidifiable upon placement in Schlemm's canal and/or through the trabecular meshwork. The solidified implant preferably has open-cell porosity and propensity of radial expansion. In a further embodiment, the implant is biocompatible and biodegradable. In still a further embodiment, the expansion of the implant or the dilation prior to implantation causes plastic deformation to Schlemm's Canal wall.

In another embodiment, an injectable foam implant is provided that is implantable within a body channel. The implant comprises injectable liquid material that is solidifiable upon placement in the body channel. The solidified implant preferably has open-cell porosity and propensity of radial expansion. In a further embodiment, the implant is biocompatible and biodegradable. In still another embodiment, the implant is loaded with a quantity of a therapeutic agent effective in treating the tissue, which is controllably released from the device into tissue of the body channel.

A method is also provided for implanting an injectable foam implant within an eye. The method may comprise creating an incision through a conjunctival tissue at a limbus and radially incising a junction between an angle tissue and sclera, which is surgically extended until Schlemm's canal is entered posteriorly. The method further comprises placing the injectable foam implant within Schlemm's canal. The implant is configured to solidify and expand after implantation for retention within the canal.

Another method for forming a trabecular foam stent. The method comprises injecting a solidifiable foam material into at least a portion of trabecular meshwork, the material solidifying following injection. Another method is described for placing a foam implant inside Schlemm's canal. The method comprises injecting a solidifiable foam material into Schlemm's canal, the material solidifying following injection.

In accordance with another method for forming a trabecular foam stent, the method comprises creating at least one opening in trabecular meshwork and injecting a solidifiable foam material into Schlemm's canal. The method further comprises introducing the foam material to the opening in trabecular meshwork and back-filling at least a portion of the opening, the material solidifying thereafter.

In accordance with one embodiment, an implant for treating glaucoma in an eye is disclosed. The implant includes an inlet section configured to be positioned in the anterior chamber of the eye and an outlet section in fluid communication with the inlet section, said outlet section configured to be positioned in Schlemm's canal of the eye. The implant comprises a hydrogel and is configured to conduct aqueous humor from the anterior chamber to Schlemm's canal.

Also disclosed is a method of treating glaucoma wherein the method includes inserting a body at least partially into at least one of the trabecular meshwork and Schlemm's canal of an eye and introducing through a lumen in said body an expandable material into at least one of the trabecular meshwork and Schlemm's canal.

In accordance with another embodiment, a kit for of treating glaucoma is disclosed having an implant configured to be positioned in an eye, said implant further configured to transmit an expandable material, through a lumen in said implant, from the anterior chamber of the eye to Schlemm's canal of the eye.

For purposes of summarizing the invention, certain embodiments, advantages, and features have been described herein. It is to be understood that not necessarily all such embodiments, advantages, or features are required in any particular embodiment. Additionally, it is to be understood that the above summary is not intended to limit in any way the embodiments, advantages, or features described below in the Detailed Description of the Preferred Embodiments or the Claims.

### BRIEF DESCRIPTION OF THE DRAWINGS

Additional objects and features of the present invention will become more apparent and the invention itself will be best understood from the following Detailed Description of the Preferred Embodiments, when read with reference to the accompanying drawings.

FIG. 1 is a sectional view of an eye for illustration purposes.

FIG. 2A is a close-up sectional view, showing the anatomical diagram of trabecular meshwork and the anterior chamber of the eye.

FIG. 2B is an enlarged cross-sectional view of an anterior chamber angle of the eye of FIG. 1 with an embodiment of an implant shown extending through trabecular meshwork from an anterior chamber to Schlemm's canal of the eye.

FIG. 3 is an embodiment of the seton implant constructed in accordance with principles of the invention.

FIG. 4 is a schematic top view of section 1-1 of FIG. 3.

FIG. 5 is another embodiment of the seton implant constructed in accordance with principles of the invention.

FIG. 6 is a perspective view illustrating a seton implant positioned within the tissue of an eye.

FIG. 7 illustrates an exemplary method for placing a seton implant at the implant site.

FIG. 8 is perspective view of an embodiment of a foam material that may be used as a foam implant.

FIG. 9 is one embodiment of the foam implant placed inside Schlemm's canal through a trabecular stent.

FIG. 10 is an embodiment of a delivery apparatus in accordance with principles of the invention.

FIG. 11 is an enlarged, cross-sectional view of an eye and demonstrating an exemplary method of implanting a porous foam implant within an eye.

### DETAILED DESCRIPTION OF THE PREFERRED EMBODIMENT

Some exemplary embodiments of the invention described below relate particularly to surgical and therapeutic treatment of glaucoma through reduction of intraocular pressure. While the description sets forth various embodiment-specific details, it will be appreciated that the description is illustrative only and should not be construed in any way as limiting the invention. Furthermore, various applications of the invention, and modifications thereto, which may occur to those who are skilled in the art, are also encompassed by the general concepts described below.

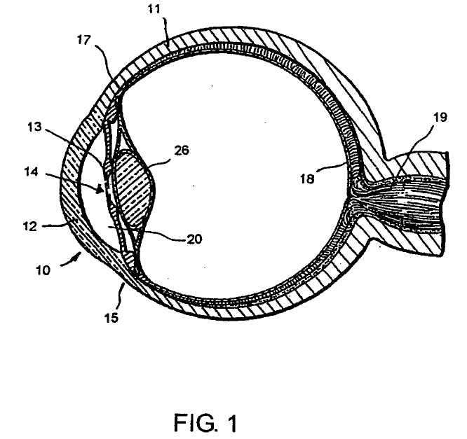

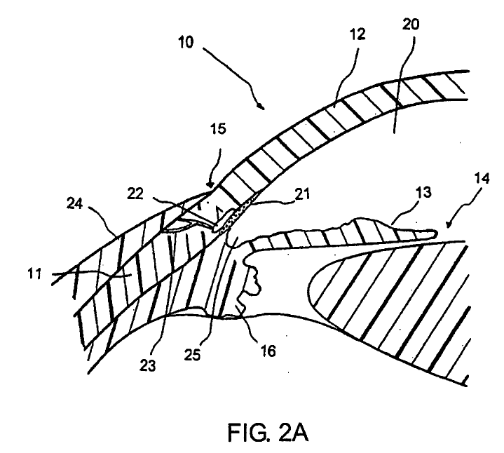

FIG. 1 is a cross-sectional view of an eye 10, while FIG. 2A is a close-up view showing the relative anatomical locations of a trabecular meshwork 21, an anterior chamber 20, and a Schlemm's canal 22. A sclera 11 is a thick collagenous tissue that covers the entire eye 10 except a portion which is covered by a cornea 12. The cornea 12 is a thin transparent tissue that focuses and transmits light into the eye and through a pupil 14, which is a circular hole in the center of an iris 13 (colored portion of the eye). The cornea 12 merges into the sclera 11 at a juncture referred to as a limbus 15. A ciliary body 16 extends along the interior of the sclera 11 and is coextensive with a choroid 17. The choroid 17 is a vascular layer of the eye 10, located between the sclera 11 and a retina 18. An optic nerve 19 transmits visual information to the brain and is the anatomic structure that is progressively destroyed by glaucoma.

The anterior chamber 20 of the eye 10, which is bound anteriorly by the cornea 12 and posteriorly by the iris 13 and a lens 26, is filled with aqueous humor (hereinafter referred to as “aqueous”). Aqueous is produced primarily by the ciliary body 16, then moves anteriorly through the pupil 14 and reaches the anterior chamber angle 25, formed between the iris 13 and the cornea 12. In a normal eye, aqueous is removed from the anterior chamber 20 through the trabecular meshwork 21. Aqueous passes through the trabecular meshwork 21 into Schlemm's canal 22 and thereafter through a plurality of aqueous veins 23, which merge with blood-carrying veins, and into systemic venous circulation. Intraocular pressure is maintained by an intricate balance between secretion and outflow of aqueous in the manner described above. Glaucoma is, in most cases, characterized by an excessive buildup of aqueous in the anterior chamber 20, which leads to an increase in intraocular pressure. Fluids are relatively incompressible, and thus intraocular pressure is distributed relatively uniformly throughout the eye 10.

As shown in FIG. 2A, the trabecular meshwork 21 is adjacent to a small portion of the sclera 11. Exterior to the sclera 11 is a conjunctiva 24. Traditional procedures that create a hole or opening for implanting a device through the tissues of the conjunctiva 24 and sclera 11 involve extensive surgery, as compared to surgery for implanting a device, as described herein, which ultimately resides entirely within the confines of the sclera 11 and cornea 12.

In a preferred embodiment, a method for increasing aqueous outflow in an eye to reduce the intraocular pressure therein comprises bypassing diseased trabecular meshwork at the level of the trabecular meshwork and thereby restoring existing outflow pathways. The method may comprise bypassing diseased trabecular meshwork at a level of said trabecular meshwork with a seton implant 31, or trabecular stent, and using existing outflow pathways. While the device 31 is referred to as seton implant, trabecular stent, trabecular shunt, device, implant, and seton implant, such designations should not be construed to limit any particular embodiment, as these designations are used interchangeably throughout. As used herein, these designations are broad terms and used in their ordinary sense and are meant to mean, without limitation, a device that is configured to conduct or permit flow of aqueous from the anterior chamber to Schlemm's canal.

The seton implant 31 may be an elongated seton or other appropriate shape, size or configuration. In one embodiment of an elongated seton implant, the seton has an inlet end, an outlet end and a lumen therebetween, wherein the inlet end is positioned at an anterior chamber of the eye and the outlet end is positioned at about an exterior surface of said diseased trabecular meshwork. Furthermore, the outlet end may be positioned into fluid collection channels of the existing outflow pathways. Optionally, the existing outflow pathways may comprise Schlemm's canal 22. In the illustrated embodiment of FIG. 2B, a trabecular stent 81 is positioned to bypass the trabecular meshwork 21 with a distal opening 87 disposed (or exposed) in Schlemm's canal 22 and a proximal opening 86 disposed (or exposed) in the anterior chamber 20. The outlet end may be further positioned into fluid collection channels up to the level of the aqueous veins with the seton inserted either in a retrograde or antegrade fashion with respect to the existing outflow pathways.

In a further alternate embodiment, a method comprises (a) creating an opening in the trabecular meshwork, wherein the trabecular meshwork comprises an interior side and exterior side; (b) inserting a seton implant into the opening; and (c) transporting the aqueous humor by said seton implant to bypass the trabecular meshwork at the level of said trabecular meshwork from the interior side to the exterior side of the trabecular meshwork.

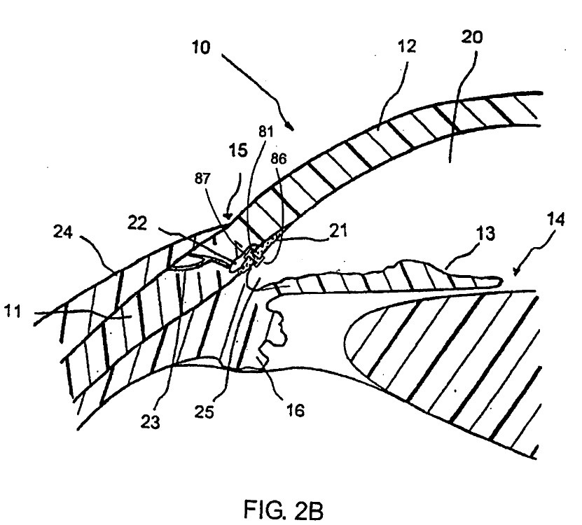

FIG. 3 shows an embodiment of the seton implant 31 constructed according to the principles of the invention. The seton implant may comprise a biocompatible material, such as a medical grade silicone, for example, the material sold under the trademark SILASTIC™, which is available from Dow Corning Corporation of Midland, Mich., or polyurethane, which is sold under the trademark PELLETHANE™, which is also available from Dow Corning Corporation. In an alternate embodiment, other biocompatible materials (biomaterials) may be used, such as polyvinyl alcohol, polyvinyl pyrolidone, collagen, heparinized collagen, tetrafluoroethylene, fluorinated polymer, fluorinated elastomer, flexible fused silica, polyolefin, polyester, polysilison, mixture of biocompatible materials, and the like. In a further alternate embodiment, a composite biocompatible material by surface coating the above-mentioned biomaterial may be used, wherein the coating material may be selected from the group consisting of polytetrafluoroethlyene (PTFE), polyimide, hydrogel, heparin, therapeutic drugs, and the like. The term “hydrogel” as used herein is a broad term and is used in its ordinary sense and is meant to cover, without limitation, any biocompatible material that increases its volume after absorbing a fluid or liquid (e.g., water).

The main purpose of the seton implant is to assist in facilitating the outflow of aqueous in an outward direction 40 into the Schlemm's canal and subsequently into the aqueous collectors and the aqueous veins so that the intraocular pressure is balanced. In one embodiment, the seton implant 31 comprises an elongated tubular element having a distal section 32 and an inlet section 44. A rigid or flexible distal section 32 is positioned inside one of the existing outflow pathways. The distal section may have a tapered outlet end 33 or a ridge 37 or other retention device protruding radially outwardly for stabilizing the seton implant inside said existing outflow pathways after implantation. For stabilization purposes, the outer surface of the distal section 32 may comprise a stubbed surface, a ribbed surface, a surface with pillars, a textured surface, or the like. The outer surface 36, including the outer region 35 and inner region 34 at the outlet end 33, of the seton implant is biocompatible and tissue compatible so that the interaction/irritation between the outer surface and the surrounding tissue is reduced. The seton implant may comprise at least one opening at a location proximal the distal section 32, away from the outlet end 33, to allow flow of aqueous in more than one direction. The opening may be located on the distal section 32 at about opposite of the outlet end 33.

In another exemplary embodiment, the seton implant 31 may have a one-way flow controlling means 39 for allowing one-way aqueous flow 40. The one-way flow controlling means 39 may be a check valve, a slit valve, a micropump, a semi-permeable membrane, or the like. To enhance the outflow efficiency, at least one optional opening 41 in the proximal portion of the distal section 32, at a location away from the outlet end 33, and in an exemplary embodiment at the opposite end of the outlet end 33, is provided.

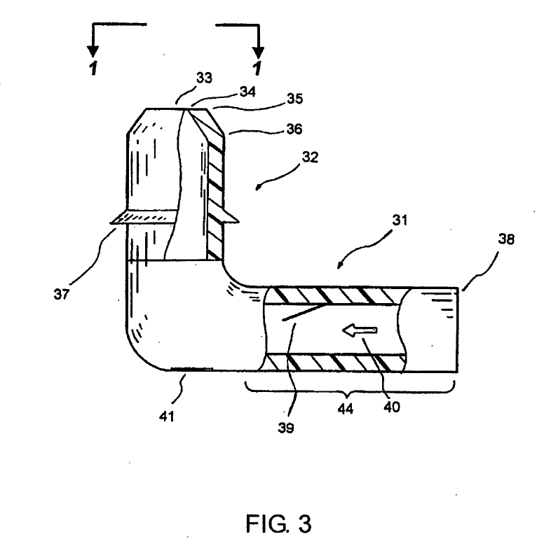

FIG. 4 shows a top schematic view of FIG. 3. The shape of the opening of the outlet end 33 and the remaining body of the distal section 32 may be oval, round or some other shape adapted to conform to the shape of the existing outflow pathways. This configuration preferably matches the contour of Schlemm's canal to stabilize the inlet section with respect to the iris and cornea by preventing rotation.

As shown in FIG. 3, the seton implant may have a length between about 0.5 mm to over about a meter, depending on the body cavity the seton implant applies to. In some embodiments, the seton implant length may be less than about 0.5 mm and greater than about a meter. The outside diameter of the seton implant may range from about 30 μm to about 500 μm, and in some embodiments, the outside diameter may be less than about 30 μm and greater than about 500 μm. The lumen diameter is preferably in the range between about 20 μm to about 150 μm. In some embodiments, the lumen diameter may be less than about 20 μm and greater than about 150 μm. The seton implant may have a plurality of lumens to facilitate multiple flow transportation. The distal section may be curved at an angle between about 30 degrees to about 150 degrees, and in an exemplary embodiment, at between about 70 to about 110 degrees, with reference to the inlet section 44. In other embodiments, the distal section may be curved at an angle less than about 30 degrees and greater than about 150 degrees with reference to the inlet section 44.

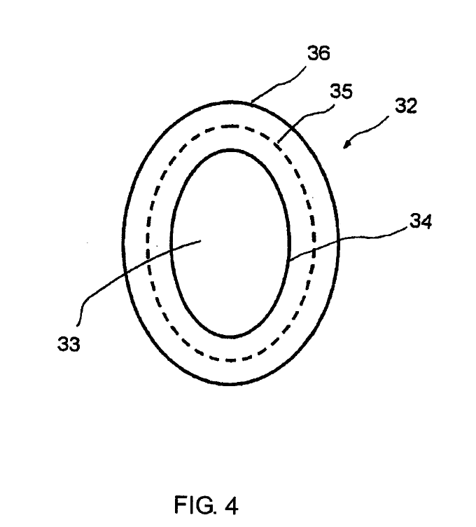

FIG. 5 shows another embodiment of the seton implant 45 constructed in accordance with the principles of the invention. In an exemplary embodiment, the seton implant 45 may comprise at least two sections: an inlet section 47 and an outlet section 46. The outlet section has an outlet opening 48 that is at the outlet end of the seton implant 45. The shape of the outlet opening 48 is preferably an oval shape to conform to the contour of the existing outflow pathways. A portion of the inlet section 47 adjacent the joint region to the outlet section 46 will be positioned essentially through the diseased trabecular meshwork while the remainder of the inlet section 47 and the outlet section 46 are outside the trabecular meshwork. As shown in FIG. 5, the long axis of the oval shape opening 48 lies in a first plane formed by an X-axis and a Y-axis. To better conform to the anatomical contour of the anterior chamber 20, the trabecular meshwork 21 and the existing outflow pathways, the inlet section 47 may preferably lie at an elevated second plane, at an angle θ, from the first plane formed by an imaginary inlet section 47A and the outlet section 46. The angle θ may be between about 30 degrees and about 150 degrees. In other embodiments, the angle θ may be less than about 30 degrees and greater than about 150 degrees.

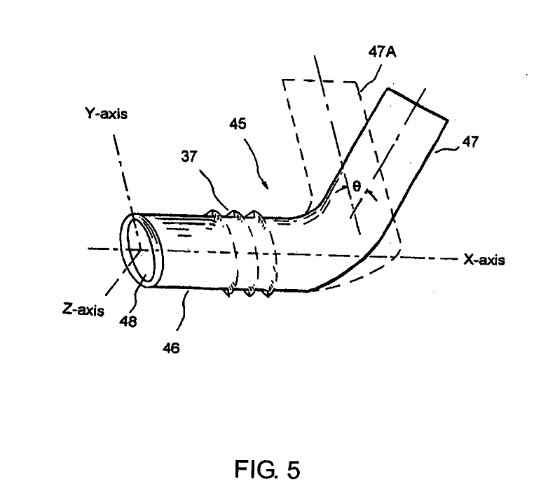

FIG. 6 shows a perspective view illustrating the seton implant 31, 45 of the present invention positioned within the tissue of an eye 10. A hole/opening is created through the diseased trabecular meshwork 21. The distal section 32 of the seton implant 31 is inserted into the hole, wherein the inlet end 38 is exposed to the anterior chamber 20 while the outlet end 33 is positioned at about an exterior surface 43 of said diseased trabecular meshwork 21. In a further embodiment, the outlet end 33 may further enter into fluid collection channels of the existing outflow pathways.

In one embodiment, the means for forming a hole or opening in the trabecular mesh 21 may comprise an incision with a microknife, an incision by a pointed guidewire, a sharpened applicator, a screw shaped applicator, an irrigating applicator, or a barbed applicator. Alternatively, the trabecular meshwork may be dissected off with an instrument similar to a retinal pick or microcurrette. The opening may alternately be created by retrogade fiberoptic laser ablation.

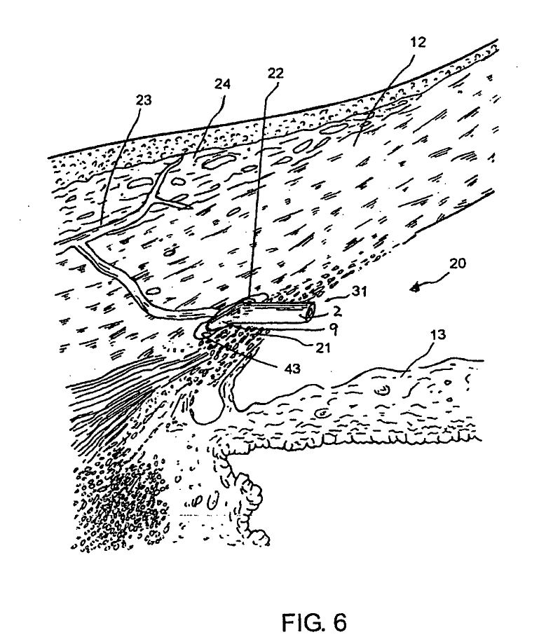

FIG. 7 shows an illustrative method for placing a seton implant at the implant site. An irrigating knife or applicator 51 comprises a syringe portion 54 and a cannula portion 55. The distal section of the cannula portion 55 has at least one irrigating hole 53 and a distal space 56 for holding a seton implant 31. The proximal end 57 of the lumen of the distal space 56 is sealed from the remaining lumen of the cannula portion 55.

For positioning the seton 31 in the hole or opening through the trabecular meshwork, the seton may be advanced over the guidewire or a fiberoptic (retrograde). In another embodiment, the seton is directly placed on the delivery applicator and advanced to the implant site, wherein the delivery applicator holds the seton securely during the delivery stage and releases it during the deployment stage.

In an exemplary embodiment of the trabecular meshwork surgery, the patient is placed in the supine position, prepped, draped and anesthesia obtained. In one embodiment, a small (less than 1 mm) self sealing incision is made. Through the cornea opposite the seton placement site, an incision is made in trabecular meshwork with an irrigating knife. The seton 31 is then advanced through the cornea incision 52 across the anterior chamber 20 held in an irrigating applicator 51 under gonioscopic (lens) or endoscopic guidance. The applicator is withdrawn and the surgery concluded. The irrigating knife may be within a size range of 20 to 40 gauges, preferably about 30 gauge.

In some embodiments, the trabecular stent 81, illustrated in FIG. 2B, operates as a “temporary stent” for introducing the injectable foam material into Schlemm's canal as a foam implant. In this embodiment, a trabecular stent 81 may be positioned with the distal opening 87 in Schlemm's canal 22 and the proximal opening 86 in the anterior chamber 20. With the trabecular stent 81 in position, injectable foam material may be inserted through the trabecular stent 81 to fill a portion of Schlemm's canal. In some embodiments, the injectable foam material may solidify, and the trabecular stent 81 may be removed following solidification of the foam material. In further embodiments, the trabecular stent 81 may be bioabsorbable and may remain in position until it is fully absorbed, leaving the solidified foam material extending through the trabecular meshwork and into Schlemm's canal.

In some embodiments, the injectable foam material is injected into Schlemm's canal with a needle, cannula, or injector. The injection route may be either through the trabecular meshwork or the sclera without a temporary stent or additional conduit. In one embodiment, an opening or slit is created in the trabecular meshwork, and injectable foam is inserted into Schlemm's canal via a needle, cannula, or injector. In some embodiments, multiple openings or slits may be created in the trabecular meshwork. In further embodiments, a portion of the injected foam material backfills a portion of the opening or slit of trabecular meshwork, and a portion of the injected foam material solidifies within the opening or slit of the trabecular meshwork.

In accordance with one method for forming a trabecular foam stent, a solidifiable foam material may be injected into a portion of trabecular meshwork, and the material may be permitted or caused to solidify thereafter. In a further embodiment, the method comprises an additional step of introducing the solidifiable foam material into Schlemm's canal prior to backfilling a portion of trabecular meshwork.

Injectable foam implants may be made of polymeric material, either a one-component liquid injectable that is solidified (via dehydration, polymerization, crosslinking, irradiation, photoinitiation, or other chemical reaction) or a two-component liquid injectable that is mixed in situ thereafter. The foaming of the implant may be associated with a gas-forming agent (e.g., carbon dioxide). Several materials may be suitable for the injectable foam implant (e.g., biodegradable or non-biodegradable), and many of these materials are described below. It will be appreciated that the listing of these materials is not exhaustive, and other materials may be used that have the same or similar properties as the materials highlighted below. Additionally, described below are methods and devices that may also be used in connection with the materials, applications, and embodiments disclosed herein.

In some embodiments, a method for treating glaucoma may be used wherein foam material is injected into Schlemm's canal, and the material comprises an effervescent medium as described in U.S. Pat. No. 6,562,374, the entire contents of which are incorporated herein by reference. The patent discloses a method for preparing biodegradable porous polymer scaffolds for tissue engineering. The method comprises the following: a) fabricating a polymer sample from a polymer solution containing at least one biodegradable polymer and an effervescent mixture; b) effervescing the polymer sample in the presence of an effervescent medium such as an aqueous alcohol solution; and c) drying. Some of the effervescent mixture used for forming pores comprises carbonate and organic acid. The effervescent mixture may comprise carbonate and organic acid that is a harmless substance to human body and that may be used in a common medicine. Carbonate is preferably selected from the group consisting of sodium bicarbonate, sodium carbonate, ammonium bicarbonate, ammonium carbonate, potassium bicarbonate, potassium carbonate, calcium carbonate, and mixtures thereof which generate carbon dioxide. Organic acid is preferably selected from the group consisting of citric acid, tartaric acid, succinic acid, maleic acid, fumaric acid, malonic acid, malic acid, gluconic acid, mucic acid, a certain amino acid, and mixtures thereof.

In some embodiments, a method may comprise injecting foam material into Schlemm's canal, wherein the foam material comprises a reticulated hyaluronic acid (SK-Gel®, manufactured by Corneal Laboratories, Paris, France). Reticulated hyaluronic acid is bioabsorbable material that may be made to any desired shape and size (three-dimensional scaffold) for tissue repair and regeneration. Non-perforating trabecular surgery (NPTS) with reticulated hyaluronic acid implant allows aqueous to leave the anterior chamber through a thin trabeculo-Descemet's membrane into a sclerocorneal space filled with SK-GEL® implant and via the outflow physiological channels. Reduction of intraocular pressure may be obtained with reduced external filtration, thus decreasing the incidence of pre- and post-operative complications related to trabeculectomy.

In some embodiments, polyacrylimide gel (T FLUX™, manufactured by IOL Tech Laboratories SA, France) may operate as a permanent implant to maintain a draining space under a superficial scleral flap created during non-penetrating glaucoma surgery. In one embodiment, the implant is made of a hydrophilic and a biocompatible acrylic material of the hydrogel family (e.g., Poly-Megma, with 38% water content). The implant may have a “T” shape consisting of a 2.75″ trunk and two arms with a combined width of 4 mm. In some embodiments, the trunk may be more or less than about 2.75″, and the combined width may be more or less than about 4 mm. In other embodiments, the implant may have shapes other than a “T”. For example, the implant may be formed in the shape of an “L” or a “J”. The implant's arms are tucked into Schlemm's canal ostiae. The implant may stabilize and provide additional support to the trabeculo-Descembet's membrane. In accordance with some embodiments, a method may comprise injecting foam material into Schlemm's canal, wherein the foam material comprises acrylic material of the hydrogel family called Poly-Megma.

In some embodiments, a method for treating glaucoma may comprise injecting foam material into Schlemm's canal, wherein the foam material comprises injectable collagen and microfibrillar collagen. For example, the STARR AQUAFLOW™ collagen implant is a lyophilized, highly purified porcine derived collagen in a cylindrical form, which may be about 5 mm wide by 4 mm long when dry. In other embodiments, other shapes and sizes may be used. For example, the width may be more or less than about 5 mm, and the length may be more or less than about 4 mm. The collagen may be a gel-like injectable material and solidified via crosslinking in situ thereafter. Injectable materials such as microfibrillar collagen and various polymeric foams may be used (see, e.g., U.S. Pat. No. 5,823,198 to Jones et al).

In one embodiment, REGEL® (manufactured by MacroMed, Sandy, Utah), a thermosensitive, biodegradable polymer/hydrogel, may be used, which are solutions at administration temperature and which become insoluble gels at body temperature. This material creates formulations which may easily be administered through small-gauge needles. Because an insoluble gel is formed immediately upon injection, the formulation tends not to expel back through the needle track and may remain at the deposited site for a period of several weeks. The gel is completely biocompatible and biodegradable, and thus, degrades into products which are well known to be metabolized and cleared. Accordingly, some embodiments include a method of treating glaucoma by injecting foam material into Schlemm's canal, wherein the foam material is a solution at administration temperature and become an insoluble gel at body temperature.

Other biomaterials may include agarose, which is a natural colloid extracted from sea weed and may be compounded with another component, such as polyacrylamide. Agarose gels may have a large pore size and may be used primarily to separate molecules with a molecule mass greater than about 200 kdal. It is contemplated that biomaterials may be used that separate molecules with a molecule mass of less than about 200 kdal. Generally, Agarose is a linear polysaccharide made up of the basic repeat unit agarobiose, which comprises alternating units of galactose and 3,6-anhydrogalactose. Another biomaterial may be hydrogels that are composed of polyacrylic acid/poly(vinyl sulfonic acid), which may have a swelling ratio in the range of about 8200-18000% at about 37° C. It is also contemplated that biomaterials may be used that have a swelling ratio less than about 8200% and greater than about 18000%.

In accordance with one method, Schlemm's canal is dilated using an infusion fluid at an elevated pressure either ab internally or ab externally. An injectable foaming material (e.g., Agarose) is then introduced into Schlemm's canal. In accordance with another method, Schlemm's canal is dilated beyond the tissue elastic yield point for permanent (i.e., plastic) deformation using an infusion fluid at an elevated pressure. The dilation may cause plastic deformation for a portion of Schlemm's canal or the aqueous collector channels in an ab interno or ab externo manner. An injectable foaming material may then be introduced into Schlemm's canal. In accordance with a further method, a foam material may be injected into Schlemm's canal, wherein the foam material comprises agarose or hydrogels composed of polyacrylic acid/poly(vinyl sulfonic acid) or a similar biomaterial which has a swelling ratio in the range of about 8200-18000% at about 37° C.

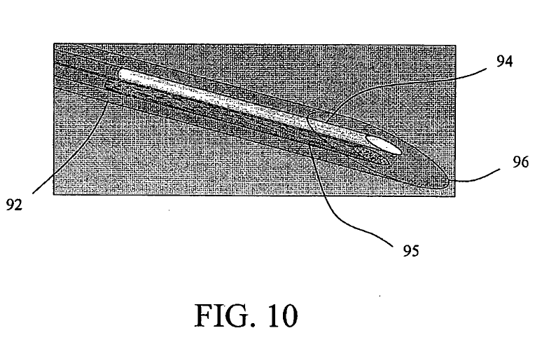

In some embodiments, the foam implant may be created by a relatively fast swelling, superporous hydrogel (e.g., vinyl monomers in the presence of gas bubbles). The injectable foam implant (or injectable foam stent) may be in a liquid state before it is injected. It may be loaded into a syringe-type injector, or applicator, that is capable of separately storing multiple ingredients and mix them during injection (such as a multi-lumen syringe or cannula). The syringe or needle 92 may have a sharp tip 96 that is configured to penetrate the cornea and the trabecular meshwork, as shown in FIG. 10. Once the syringe or needle 92 is inside Schlemm's canal, the liquid foam ingredients may be expelled from the applicator. As the ingredients mix, the chemical reaction creates tiny gas bubbles, and these bubbles form the pores or voids in the foam, preferably open-cell pores. In further embodiments, a foam-forming agent may be added in the injectable hydrogel. In these embodiments, expansion occurs as the foam sets and absorbs aqueous. The setting process may be designed and configured to take less than a few minutes in some embodiments, but in other embodiments, the setting process preferably takes less than about 1 minute. It is contemplated that the setting process may take more than about a few minutes in yet other embodiments. In some embodiments, multiple foam stents may be injected without reloading the applicator. In one embodiment, the combination of porosity and stent expansion does not impede aqueous flow characteristics within Schlemm's Canal acutely and chronically.

Several advantages may be realized by using an injectable foam implant, as described herein. For example, the retention strength may be high if the foam implant extends along Schlemm's canal. Additionally, multiple implant deliveries may be achieved by serially stacking the implant or stent in the applicator, and because the implant is delivered as a liquid, the applicator or injector may have a relatively small diameter. Using a foam implant reduces or eliminates rotational orientation during the implanting procedure. Further, the open-cell structure of the implant or stent may provide multiple outflow paths for aqueous. The implant may also provide support to open Schlemm's canal and assist in keeping it open.

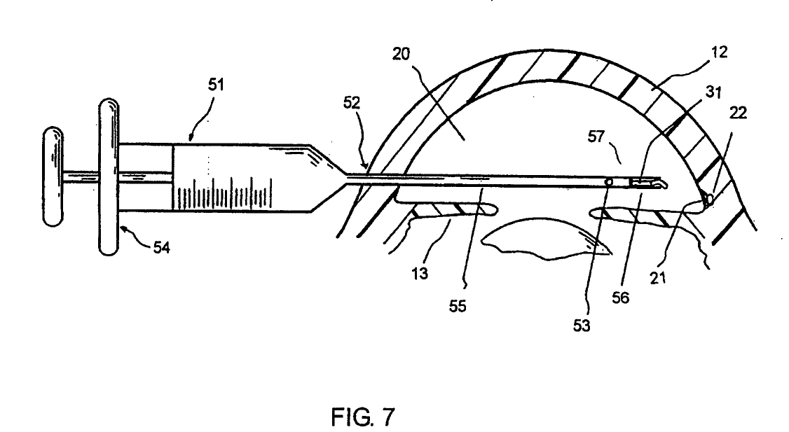

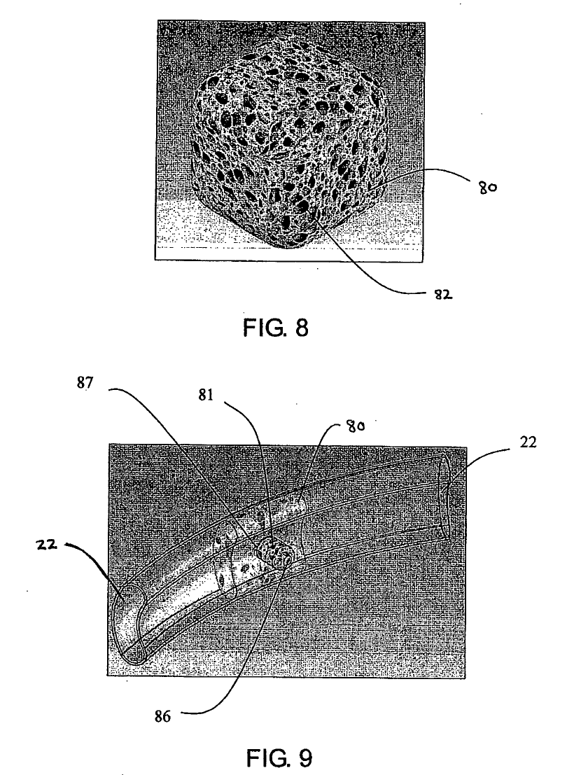

FIG. 8 shows a perspective view of one embodiment of foam material that may be used as a foam implant 80, and FIG. 9 shows one embodiment of the foam implant 80 placed inside Schlemm's canal 22. In one preferred embodiment, the foam implant comprises a plurality of pores 82. Preferably, most of the pores 82 are open-cell pores that connect to other pores and to the exterior of the implant. In one embodiment, a temporary hollow stent (or a hollow conduit/passageway) is used as conduit for injecting the liquid foaming material.

In another embodiment, the injectable foaming material is injected via a syringe or needle 92, as shown in FIG. 10, through an opening of the trabecular meshwork 21 into Schlemm's canal 22. In one embodiment, the injectable material is a one-component liquid that passes through the conduit 94. Once the injectable material is disposed in Schlemm's canal, a fiber optic cable 95 may be deployed to a location adjacent the material, enabling the fiber optic cable 95 to emit photodynamic light for crosslinking the material to solidify the implant in situ.

In another embodiment, the injectable material is comprises multiple-components and is injected through lumens of a plurality of conduits 94 within the needle 92. In some embodiments, the two conduits 94, 95 may comprise components that react when combined to form a foaming material. Thus, when a distal tip 96 of the needle is positioned through the trabecular meshwork 21 and into Schlemm's canal 22, the components are injected into Schlemm's canal 22 and react to form an implant, such as that illustrated in FIG. 9. The needle, syringe, or foam injector may be coated with bioactive agents, such as heparin, antibiotic, anti-infective, anti-inflammatory and the like.

In accordance with some embodiments, the injectable foaming material comprises an occlusive agent that precipitates on contact with water or water-containing liquids and certain foam-forming agent. Such agents are described in U.S. patent application Ser. No. 10/038,730, the entire contents of which are incorporated herein by reference, discloses an occlusive agent which may be made from a precursor composition containing at least one biodegradable polymeric component. The biodegradable polymer is selected from biodegradable polyesters, polyglycolic acids, polylactic acids, polycaprolactone, and their copolymers and copolymers with trimethylene carbonate, polyhydroxybutyrate and polyhydroxyvalerate and their copolymers, and polyanhydrides. The polymeric mixture or occlusive precursor precipitates on contact with water, or water-containing liquids such as blood, in the body to form an occlusive mass. In some embodiments, the solvent is ethanol because dilute ethanol has minor toxic or harmful effects to the human body when compared to other organic solvents. In other embodiments, other solvents may be used that have similar effects on the body, and in some embodiments, although not necessarily preferred, solvents may be used that have greater toxic or harmful effects on the body than ethanol. In the precipitation method, the polymer may be dissolved in a solvent that is miscible with blood, and upon contact with blood, the solvent may be diluted and the water-insoluble polymer precipitates. One such precipitative material that may be used in this manner is polyvinyl acetate (PVAc), which is solvable in an ethanol/water mixture.

In some embodiments, an injectable foaming material may be used that comprises a polymeric composition and a foam-forming agent. The polymeric composition may comprise water insoluble, non-crosslinked polymeric compounds dissolved in a polar, non-toxic water miscible solvent. The polymeric composition preferably solidifies when placed in contact with living tissue by absorption of water, as described in U.S. Pat. No. 4,631,188, the entire contents of which are incorporated herein by reference. The patent discloses a non-toxic physiologically-acceptable polymeric composition comprised of water insoluble, non-crosslinked polymeric compounds dissolved in a polar, non-toxic water miscible solvent. The polymeric composition preferably solidifies when placed in contact with living tissue by absorption of water and by gradual release of solvent into the surrounding tissue. The polymeric composition may be selected from the group consisting of polymers and copolymers of acrylonitrile, polyvinylacetate, poly(vinylacetate-co-venylalcohol), 2-hydroxyethylacrylate, methylacrylate, poly (n-vinyliminocarbonyl), polycondensates, polyadducts acrylamide, N-substituted acrylamide, acrylhydrazide, N-substituted acrylhydrazide, acrylic acid, acrylic acid salts, glutarimide and vinylsulfonate. The polymeric compound is admixed with the solvent in a concentration of from about 0.1 to about 50% by weight, such that the resulting polymeric solution when in contact with water forms an integral solid (homogeneous or porous) via coagulation process.

In additional embodiments, an injectable foaming material may be used that comprises soluble or fibrillar collagen and a foam-forming agent. The collagen is preferably polymerized into a soft gel by exposure to air, light, an initiator, or an oxidative enzyme, as described in U.S. Pat. No. 5,476,515, the entire contents of which are incorporated herein by reference. This patent discloses purified soluble or partially fibrillar collagen modified with acylating agents, sulfonating agents or combinations thereof to form a clear transparent collagen composition. The modified collagen may be injected into a lens capsular sac or Schlemm's canal to form an implant in situ that is resistant to epithelialization. The collagen-based implant produced by such a method may remain in its original viscous liquid state or may be polymerized into a soft gel. Further, the modified collagen composition is polymerized by exposing the composition to air, light, an initiator, or an oxidative enzyme, wherein the initiator is selected from the group consisting of sodium persulfate, sodium thiosulfate, ferrous chloride tetrahydrate, sodium bisulfite and a combination thereof.

Some embodiments contemplate an injectable foaming material that comprises an occluding agent and a foam-forming agent, wherein the occluding agent hardens when reacts to catalyst or radiation of a certain wavelength, as described in U.S. Pat. No. 5,795,331, the entire contents of which are incorporated herein by reference. The patent discloses an occluding agent preferably comprising liquid or solid materials or objects that effect occlusion through a variety of reactions. The occluding agent is preferably introduced as a liquid and hardens within the aneurysm chamber. In one embodiment, the occluding agent may require a reactive catalyst to harden, and the balloon catheter may be provided with a further lumen and exit port adjacent to the first exit port and a further delivery lumen for separately introducing the catalyst. In a further embodiment, the occluding agent may react to radiation of a certain wavelength which is provided from an external source and introduced into the distal segment of the balloon catheter and directed at the opening to effect hardening of the delivered occluding agent by a light conductor or optical fiber.

In some embodiments, an injectable foaming material may be used that comprises a stretch-crystallizable shape-transformable elastomeric material and a foam-forming agent. The injectable canal implant may be formed of stretch-crystallizable silicone elastomers formulated to stretch crystallize at near ambient temperatures upon elongations greater than 100% and to recover their original configuration immediately upon exposure to body temperature following implantation, as described in U.S. Pat. No. 6,030,416, the entire contents of which are incorporated herein by reference. The patent discloses a stretch-crystallizable shape-transformable elastomeric material formulated to exhibit the property of stretch crystallization upon substantial elongation of the implant to form stable, small-incision implant configurations having at least one dimension substantially reduced for insertion through a surgical incision that is small relative to the incision size necessary to implant the unstretched implant. Exemplary embodiments include intraocular implants formed of optically clear, high refractive index stretch-crystallizable silicone elastomers formulated to stretch crystallize at near ambient temperatures upon elongations greater than or equal to 300% and to recover their original configuration immediately upon exposure to body temperature following implantation.

In yet additional embodiments, an injectable foaming material comprises a polysiloxane copolymer compound and a foam-forming agent. The polysiloxane copolymer with a photoinitiator or a crosslinking agent is capable of being photopolymerized into a solid foaming implant in situ, as described in U.S. Pat. No. 6,399,734, the entire contents of which are incorporated herein by reference. The patent discloses an injectable lens material having suitable viscosity for being injected through standard cannula and comprising a mixture of a polysiloxane copolymer, a photoinitiator, and, optionally, a crosslinking agent. The polysiloxane copolymer is preferably capable of being photopolymerized into a solid intraocular lens and has functional acryl groups at terminal ends of the copolymer, a refractive index that is suitable for restoring the refractive power of the natural crystalline lens, and siloxane monomer units in the polysiloxane copolymer selected from the group consisting of substituted and unsubstituted arylsiloxanes, substituted and unsubstituted arylalkylsiloxanes, and substituted and unsubstituted alkyl(alkyl)siloxanes, and mixtures thereof. Preferably, at least one of the siloxane monomer units is substituted with one or more fluorine atoms, and wherein at least one siloxane monomer unit is an arylsiloxane or an arylalkylsiloxane.

In further embodiments, an injectable foaming material comprises a hydrogel forming aqueous composition of water soluble polymers and a foam-forming agent. The hydrogel forming aqueous composition is preferably capable of being photoinitiated crosslinking to form a solid foaming implant in situ, as described in U.S. Pat. No. 6,747,090, the entire contents of which are incorporated herein by reference. The patent discloses a hydrogel forming aqueous composition of water soluble polymers having a sufficiently high coherency that it substantially is not dispersed when being injected into an aqueous environment of a body site such as the capsular bag of the eye to undergo a photoinitiated crosslinking reaction.

In some embodiments, a device is provided (for example, a trabecular stent or a temporary hollow conduit) that is placed within an eye. The device may comprise an inlet section having at least one inlet lumen and an outlet section having at least one outlet lumen. The device preferably is sized and configured to permit fluid or injectable foaming material entering the at least one inlet lumen and then exit through at least one of the outlet lumens. One terminal of the temporary hollow conduit may be placed generally inside the anterior chamber while the terminal of the outlet section is placed generally inside Schlemm's canal.

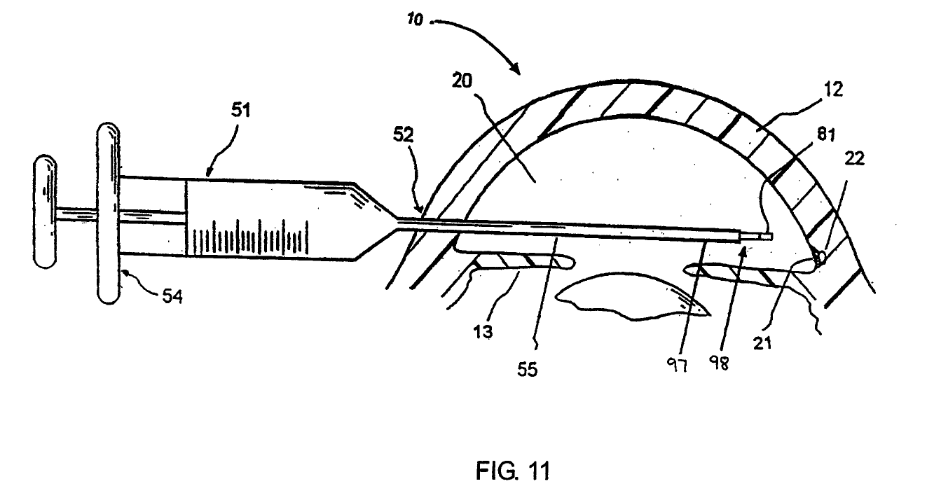

FIG. 11 shows an illustrative method for placing an implant at the implant site. An irrigating knife or applicator 51 comprises a syringe portion 54 and a cannula portion 55. The distal section 97 of the cannula portion 55 has a distal tip 98 for injecting foaming material through the implant 81 into Schlemm's canal. The implant 81 is preferably detachably connected to the distal tip 98, thus permitting the implant 81 to be detached from the distal tip 98 following injection of the foaming material into Schlemm's canal.

For positioning the implant 81 through the trabecular meshwork, the implant may be advanced on the distal tip 98 of the applicator 51. The syringe portion 54 may be advanced through a corneal incision 52 and across the anterior chamber 20. The implant 81 is brought to a position adjacent the trabecular meshwork 21, and the implant 81 is advanced through the trabecular meshwork 21. Preferably, the implant 81 is self-trephining, thus permitting the implant 81 to be advanced through the trabecular meshwork 21. The trabecular meshwork can also be breached with a separate trephining tool or a tip having thermal energy or cryogenic energy. When positioned, implant 81 preferably extends through the trabecular meshwork and into Schlemm's canal 22. With the implant 81 in this position, the foaming material, or other material described herein, may be injected from the distal tip 98 through the implant 81 into Schlemm's canal 22. Following injection of the material into Schlemm's canal 22, the implant 81 may be detached from the distal tip 98 and remain in the trabecular meshwork 21. In other embodiments, following injection of the material into Schlemm's canal 22, the applicator 51 may be configured to permit relocation of the distal tip 98 through the trabecular meshwork 21 at multiple locations to apply multiple injections of the material. For example, the applicator 51 may be configured to permit advancement through the trabecular meshwork 21 in multiple locations of the trabecular meshwork 21 to provide injections of the material through the distal tip 98 into Schlemm's canal without requiring removal of the applicator 51 from the corneal incision 52.

In some embodiments, the trabecular stent 81, illustrated in FIGS. 9 and 11, may operate as a “temporary stent” for introducing the injectable foam material into Schlemm's canal as a foam implant. In this embodiment, the trabecular stent 81 may be positioned with the distal opening 87 in Schlemm's canal 22 and the proximal opening 86 in the anterior chamber 20. With the trabecular stent 81 in position, injectable foam material may be inserted through the trabecular stent 81 to fill a portion of Schlemm's canal. In some embodiments, the injectable foam material may solidify, and the trabecular stent 81 may be removed following solidification of the foam material. In further embodiments, the trabecular stent 81 may be bioabsorbable and may remain in position until it is fully absorbed, leaving the solidified foam material extending through the trabecular meshwork and into Schlemm's canal. In yet other embodiments, the trabecular stent 81 may be removed following injection of the material.

In some embodiments, the injectable foam material is injected into Schlemm's canal with a needle, cannula, or injector. The injection route may be either through the trabecular meshwork or the sclera without a temporary stent or additional conduit. In one embodiment, an opening or slit is created in the trabecular meshwork, and injectable foam is inserted into Schlemm's canal via a needle, cannula, or injector. In some embodiments, multiple openings or slits may be created in the trabecular meshwork. In further embodiments, a portion of the injected foam material backfills a portion of the opening or slit of trabecular meshwork, and a portion of the injected foam material solidifies within the opening or slit of the trabecular meshwork.

In another embodiment, the device is coated with at least one polymer film that contains at least one therapeutic agent or substance. The polymer film preferably permits delivery of the therapeutic substance to ocular tissues over time. In one embodiment, the polymer film is a hydrogel. In still another embodiment, the device is made of a material comprising a polymer base. The polymer base may be selected from the group consisting of silicone, polyurethane, PMMA, acrylic, poly(lactic acid), polyethylene-vinyl acetate, poly(lactic-co-glycolic acid), poly(D,L-lactide), poly(D,L-lactide-co-trimethylene carbonate), collagen, heparinized collagen, poly(caprolactone), poly(glycolic acid), and copolymer. The device may also be made of metal, such as Nitinol, stainless steel, titanium or precious metals.

In some embodiments, the implant may comprise a biocompatible material that hydrates and expands after implantation, permitting the implant to be locked into position around the trabecular meshwork opening or inside Schlemm's canal. For example, such a material is described in copending U.S. patent application Ser. No. 09/549,350, the entire contents of which are incorporated herein by reference. The implant may be of porous material, semi-rigid material, soft material, hydrophilic material, hydrophobic material, hydrogel, elastic material, and the like.

In accordance with some embodiments, a porous hydrogel material may be used as an implant by creating gas pockets in the gel and then removing this gas. Removal of the gas creates a porous material, and the initial incorporation of sufficient gas permits creation of a material with an open, interconnected pore structure. Another advantage of this material is that the pore structure may be maintained over extended time periods and that the gels may maintain a high mechanical integrity that allows cell penetration and proliferation without destruction or compression of the material. The approach is in contrast to other processing approaches typically used to achieve a porous structure with these types of materials (e.g., lyophilization), in which the porous nature is lost as the material rehydrates and/or the material is significantly weakened by the process.

One of the advantages of trabecular bypass surgery, as disclosed herein in numerous embodiments, and the use of an implant to bypass diseased trabecular meshwork at the level of trabecular meshwork and thereby use existing outflow pathways is that the treatment of glaucoma is substantially simpler than in existing therapies. Additionally, the simple microsurgery may be performed on an outpatient basis with rapid visual recovery and greatly decreased morbidity. Finally, a distinct approach is used than is found in existing implants. Physiological outflow mechanisms are used or re-established by the implant of the present invention, in contradistinction with previously disclosed methodologies.

Although embodiments of the present invention have been disclosed in the context of certain preferred embodiments and examples, it will be understood by those skilled in the art that the present invention extends beyond the specifically disclosed embodiments to other alternative embodiments and/or uses of the present invention and obvious modifications and equivalents thereof. In addition, while a number of variations of the present invention have been shown and described in detail, other modifications, which are within the scope of present invention, will be readily apparent to those of skill in the art based upon this disclosure. It is also contemplated that various combinations, subcombinations, or cross-applications of the specific features and aspects of the embodiments may be made and still fall within the scope of the present invention. Accordingly, it should be understood that various features and aspects of the disclosed embodiments can be combined with or substituted for one another in order to form varying modes of the present invention. Thus, it is intended that the scope of the present invention herein disclosed should not be limited by the particular disclosed embodiments described above, but should be determined only by a fair reading of the claims that follow.

1. An implant for treating glaucoma in an eye, the implant comprising:
an inlet section configured to be positioned in the anterior chamber of the eye; and
an outlet section in fluid communication with the inlet section, said outlet section configured to be positioned in Schlemm's canal of the eye;
wherein the implant comprises a hydrogel and is configured to conduct aqueous humor from the anterior chamber to Schlemm's canal.

2. A method of treating glaucoma comprising:
inserting a body at least partially into at least one of the trabecular meshwork and Schlemm's canal of an eye; and
introducing through a lumen in said body an expandable material into at least one of the trabecular meshwork and Schlemm's canal.

3. The method of claim 2, further comprising expanding Schlemm's canal with said material.

4. The method of claim 2, further comprising hardening said material after it is introduced into Schlemm's canal.

5. The method of claim 2, wherein said material is introduced into Schlemm's canal as a liquid.

6. The method of claim 5, further comprising solidifying said liquid.

7. The method of claim 6, wherein said solidifying comprises photoactivating said material.

8. The method of claim 6, wherein said solidifying comprises a method from the group consisting of dehydration, polymerization, crosslinking, irradiation, and photoinitiation.

9. The method of claim 2, wherein said material comprises a hydrogel.

10. The method of claim 2, wherein said material comprises a polymer.

11. The method of claim 2, wherein said material comprises a collagen.

12. The method of claim 11, wherein the material comprises microfibrillar collagen.

13. The method of claim 2, wherein said material comprises a colloid.

14. The method of claim 2, wherein the material comprises an effervescent medium.

15. The method of claim 2, wherein the material comprises reticulated hyaluronic acid.

16. The method of claim 2. wherein the material comprises acrylic.

17. The method of claim 2, wherein the material comprises agarose.

18. The method of claim 2, wherein the material comprises polyacrylic acid.

19. The method of claim 2, wherein the material comprises water insoluble, non-crosslinked polymeric compounds dissolved in a polar, non-toxic water miscible solvent.

20. The method of claim 2, wherein the material comprises a stretch-crystallizable shape-transformable elastomeric material.

21. The method of claim 2, wherein the material comprises a polysiloxane copolymer compound with a photoinitiator or a crosslinking agent that enables photopolymerizing said compound.

22. The method of claim 2, wherein the material is introduced through the trabecular meshwork

23. The method of claim 2, wherein the material is introduced through sclera tissue.

24. A kit for of treating glaucoma, comprising:
an implant configured to be positioned in an eye, said implant further configured to transmit an expandable material, through a lumen in said implant, from the anterior chamber of the eye to Schlemm's canal of the eye;
the expandable material.

25. The kit of claim 24, fit comprising a sterile package that contains said implant and said expandable material.

26. The kit of claim 24, wherein said material comprises a hydrogel.

27. The kit of claim 24, wherein said material comprises a polymer.

28. The kit of claim 24, wherein said material comprises a collagen.

29. The kit of claim 24, wherein said material comprises a colloid.

30. The kit of claim 24, wherein the material comprises an effervescent medium.

31. The kit of claim 24, wherein the material comprises reticulated hyaluronic acid.

32. The kit of claim 24, wherein the material comprises acrylic.

33. The kit of claim 24, wherein the material comprises agarose.

34. The kit of claim 24, wherein the material comprises polyacrylic acid.

35. A method of treating glaucoma comprising:
inserting a body at least partially into at least one of the trabecular meshwork and Schlemm's canal of an eye; and
introducing through a lumen in said body a hardenable material into at least one of the trabecular meshwork and Schlemm's canal.

36. The method of claim 35, wherein said material comprises a polymer.

37. A kit for of treating glaucoma, comprising:
an implant configured to be positioned in an eye, said implant further configured to transmit a hardenable material, through a lumen in said implant, from the anterior chamber of the eye to Schlemm's canal of the eye;
the hardenable material.

38. The kit of claim 37, wherein said material comprises a polymer.
